SEQUENCE LISTING (17 sequences)

SEQ ID NO: 1 - DNA, 13753 bp, Artificial Sequence
  lengthened PR transcript
ggatccattttataagctcaaagataattacttttcagactaagaatatttagggtaaaaagtactgttcaacatctctactgaggatgttatgatgtagcacactctataagctggagctaaaggaaactttccttaaagtgctatttactaaaaattggaacacattccttaagacaaatcgaagtgtggcacacaacatccaaacttccatcatagatacagaggtgttaccatctcccactcccaaatttctttgtcacgctgaggatactcaagaggagcaggacatgttggtcgcagcaggagaaacttgaaagcattcacttttatggaactcataagggagagaatctcttatttagtatcgtccttgatacatttattattttaaaagataatgtagccaaatgtcttcctctgtgttaaatctttacaaaactgaaatcttaaaatggtgacaaaaattctacttctgatagaatctattcatttttccaattagatagggcataattcttaatttgcaaaacaaaacgtaatatgcttatgaggttccatcccaaagaacctgctattgagagtagcattcagaataacgggtggaaatgccaactccagagtttcagatcctaccggtaattggggtagggaggggctttgggcggggcctccctagaggaggaggcgttgttagaaagctgtctggccagtccacagctgtcactaatcggggtaagccttgttgtatttgcgcgtgtgggtggcattctcaatgagaactagcttcacttgtcatttgagtgaaatctacaacccgaggcggctagtgctcccgcactactgggatctgagatcttcggagatgactgtcgcccgcagtacggagccagcagaagtccgacccttcctgggaatgggctgtaccgagaggtccgactagccccagggttttagtgagggggcagtggaactcagcgagggactgagagcttcacagcatgcacgagtttgatgccagagaaaaagtcgggagataaaggagccgcgtgtcactaaattgccgtcgcagccgcagccactcaagtgccggacttgtgagtactctgcgtctccagtcctcggacagaagttggagaactctcttggagaactccccgagttaggagacgagatctcctaacaattactactttttcttgcgctccccacttgccgctcgctgggacaaacgacagccacagttcccctgacgacaggatggaggccaagggcaggagctgaccagcgccgccctcccccgcccccgacccaggaggtggagatcctccggtccagccacattcaacacccactttctcctccctctgcccctatattcccgaaaccccctcctccttcccttttccctcctccctggagacgggggaggagaaaaggggagtccagtcgtcatgactgagctgaaggcaaagggtccccgggctccccacgtggcgggcggcccgccctcccccgaggtcggatccccactgctgtgtcgcccagccgcaggtccgttcccggggagccagacctcggacaccttgcctgaagtttcggccatacctatctccctggacgggctactcttccctcggccctgccagggacaggacccctccgacgaaaagacgcaggaccagcagtcgctgtcggacgtggagggcgcatattccagagctgaagctacaaggggtgctggaggcagcagttctagtcccccagaaaaggacagcggactgctggacagtgtcttggacactctgttggcgccctcaggtcccgggcagagccaacccagccctcccgcctgcgaggtcaccagctcttggtgcctgtttggccccgaacttcccgaagatccaccggctgcccccgccacccagcgggtgttgtccccgctcatgagccggtccgggtgcaaggttggagacagctccgggacggcagctgcccataaagtgctgccccggggcctgtcaccagcccggcagctgctgctcccggcctctgagagccctcactggtccggggccccagtgaagccgtctccgcaggccgctgcggtggaggttgaggaggaggatagctctgagtccgaggagtctgcgggtccgcttctgaagggcaaacctcgggctctgggtggcgcggcggctggaggaggagccgcggcttgtccgccgggggcggcagcaggaggcgtcgccctggtccccaaggaagattcccgcttctcagcgcccagggtcgccctggtggagcaggacgcgccgatggcgcccgggcgctccccgctggccaccacggtgatggatttcatccacgtgcctatcctgcctctcaatcacgccttattggcagcccgcactcggcagctgctggaagacgaaagttacgacggcggggccggggctgccagcgcctttgccccgccgcggacttcaccctgtgcctcgtccaccccggtcgctgtaggcgacttccccgactgcgcgtacccgcccgacgccgagcccaaggacgacgcgtaccctctctatagcgacttccagccgcccgctctaaagataaaggaggaggaggaaggcgcggaggcctccgcgcgctccccgcgttcctaccttgtggccggtgccaaccccgcagccttcccggatttcccgttggggccaccgcccccgctgccgccgcgagcgaccccatccagacccggggaagcggcggtgacggccgcacccgccagtgcctcagtctcgtctgcgtcctcctcggggtcgaccctggagtgcatcctgtacaaagcggagggcgcgccgccccagcagggcccgttcgcgccgccgccctgcaaggcgccgggcgcgagcggctgcctgctcccgcgggacggcctgccctccacctccgcctctgccgccgccgccggggcggcccccgcgctctaccctgcactcggcctcaacgggctcccgcagctcggctaccaggccgccgtgctcaaggagggcctgccgcaggtctacccgccctatctcaactacctgaggccggattcagaagccagccagagcccacaatacagcttcgagtcattacctcagaagatttgtttaatctgtggggatgaagcatcaggctgtcattatggtgtccttacctgtgggagctgtaaggtcttctttaagagggcaatggaagggcagcacaactacttatgtgctggaagaaatgactgcatcgttgataaaatccgcagaaaaaactgcccagcatgtcgccttagaaagtgctgtcaggctggcatggtccttggaggtcgaaaatttaaaaagttcaataaagtcagagttgtgagagcactggatgctgttgctctcccacagccattgggcgttccaaatgaaagccaagccctaagccagagattcactttttcaccaggtcaagacatacagttgattccaccactgatcaacctgttaatgagcattgaaccagatgtgatctatgcaggacatgacaacacaaaacctgacacctccagttctttgctgacaagtcttaatcaactaggcgagaggcaacttctttcagtagtcaagtggtctaaatcattgccaggttttcgaaacttacatattgatgaccagataactctcattcagtattcttggatgagcttaatggtgtttggtctaggatggagatcctacaaacatgtcagtgggcagatgctgtattttgcacctgatctaatactaaatgaacagcggatgaaagaatcatcattctattcattatgccttaccatgtggcagatcccacaggagtttgtcaagcttcaagttagccaagaagagttcctctgtatgaaagtattgttacttcttaatacaattcctttggaagggctacgaagtcaaacccagtttgaggagatgaggtcaagctacattagagagctcatcaaggcaattggtttgaggcaaaaaggagttgtgtcgagctcacagcgtttctatcaacttacaaaacttcttgataacttgcatgatcttgtcaaacagcttcatctgtactgcttgaatacatttatccagtcccgggcactgagtgttgaatttccagaaatgatgtctgaagttattgctgcacaattacccaagatattggcagggatggtgaaaccccttctctttcataaaaagtgaatgtcatctttttcttttaaagaattaaattttgtggcatgtctttttgttttggtcaggattatgaggtcttgagtttttataatgttcttctgaaagccttacatttataacatcatagtgtgtaaatttaaaagaaaaattgtgaggttctaattattttcttttataaagtataattagaatgtttaactgttttgtttacccatattttcttgaagaatttacaagattgaaaaagtactaaaattgttaaagtaaactatatcttatccatattatttcataccatgtaggtgaggatttttaacttttgcatctaacaaatcatcgacttaagagaaaaaatcttacatgtaataacacaaagctattatatgttatttctaggtaactccctttgtgtcaattatatttccaaaaatgaacctttaaaatggtatgcaaaattttgtctatatatatttgtgtgaggaggaaattcataactttcctcagattttcaaaagtatttttaatgcaaaaaatgtagaaagagtttaaaaccactaaaatagattgatgttcttcaaactaggcaaaacaactcatatgttaagaccattttccagattggaaacacaaatctcttaggaagttaataagtagattcatatcattatacaaatagtattgtgggttttgtaggtttttaaaataaccttttttggggagagaattgtcctctaatgaggtattgcgagtggcacataagaaatcagaagattatggcctaactgtactccttaccaactgtggcatgctgaaagttagtcactcttactgattctcaattctctcacctttgaaagtagtaaaatatctttcctgccaattgctcctttgggtcagagcttattaacatcttttcaaatcaaaggaaagaagaaagggagaggaggaggagggaggtatcaattcacatacctttctcctctttatcctccactatcatgaattcatattatgtttcagccatgcaaatctttttaccatgaaatttcttccagaattttccccctttgacacaaattccatgcatgtttcaaccttcgagactcagccaaatgtcatttctgtaaaatcttccctgagtcttccaagcagtaatttgccttctcctagagtttacctgccattttgtgcacatttgagttacagtagcatgttattttacaattgtgactctcctgggagtctgggagccatataaagtggtcaatagtgtttgctgactgagagttgaatgacattttctctctgtcttggtattactgtagatttcgatcattctttggttacatttctgcatatttctgtacccatgactttatcactttcttctcccatgctttatctccatcaattatcttcattacttttaaattttccacctttgcttcctactttgtgagatctctccctttactgactataacatagaagaatagaagtgtattttatgtgtcttaaggacaatactttagattccttgttctaagtttttaaactgaatgaatggaatattatttctctccctaagcaaaattccacaaaacaattatttcttatgtttatgtagccttaaattgttttgtactgtaaacctcagcataaaaactttcttcatttctaatttcattcaacaaatattgattgaatacctggtattagcacaagaaaaatgtgctaataagccttatgagaatttggagctgaagaaagacatataactcaggaaagttacagtccagtagtaggtataaattacagtgcctgataaataggcattttaatatttgtacactcaacgtatactaggtaggtgcaaaacatttacatataattttactgatacccatgcagcacaaaggtactaactttaaatattaaataacacctttatgtgtcagtaattcatttgcattaaatcttattgaaaaggctttcaatatattttccccacaaatgtcatcccaagaaaaaagtatttttaacatctcccaaatataatagttacaggaaatctacctctgtgagagtgacacctctcagaatgaactgtgtgacacaagaaaatgaatgtaggtctatccaaaaaaaaccccaagaaacaaaaacaatattattagccctttatgcttaagtgatggactcagggaacagttgatgttgtgatcattttattatctgattcttgttactttgaattaaaccaatattttgatgatataaatcatttccaccagcatatatttaatttccataataactttaaaattttctaatttcactcaactatgagggaatagaatgtggtggccacaggtttggcttttgttaaaatgtttgatatcttcgatgttgatctctgtctgcaatgtagatgtctaaacactaggatttaatatttaaggctaagctttaaaaataaagtacctttttaaaaagaatatggcttcaccaaatggaaaatacctaatttctaaatctttttctctacaaagtcctatctactaatgtctccattactatttagtcatcataaccattatcttcattttacatgtcgtgttctttctggtagctctaaaatgacactaaatcataagaagacaggttacatatcaggaaatacttgaaggttactgaaatagattcttgagttaatgaaaatattttctgtaaaaaggtttgaaaagccatttgagtctaaagcattatacctccattatcagtagttatgtgacaattgtgtgtgtgtttaatgtttaaagatgtggcactttttaataaggcaatgctatgctattttttcccatttaacattaagataatttattgctatacagatgatatggaaatatgatgaacaatattttttttgccaaaactatgccttgtaagtagccatggaatgtcaacctgtaacttaaattatccacagatagtcatgtgtttgatgatgggcactgtggagataactgacataggactgtgccccccttctctgccacttactagctggatgagattaagcaagtcatttaactgctctgattaaacctgcctttcccaagtgctttgtaatgaatagaaatggaaaccaaaaaaaacgtatacaggccttcagaaatagtaattgctactattttgttttcattaagccatagttctggctataattttatcaaactcaccagctatattctacagtgaaagcaggattctagaaagtctcactgttttatttatgtcaccatgtgctatgatatatttggttgaattcatttgaaattagggctggaagtattcaagtaatttcttctgctgaaaaaatacagtgttttgagtttagggcctgttttatcaaagttctaaagagcctatcactcttccattgtagacattttaaaataatgacactgattttaacatttttaagtgtctttttagaacagagagcctgactagaacacagcccctccaaaaacccatgctcaaattatttttactatggcagcaattccacaaaagggaacaatgggtttagaaattacaatgaagtcatcaacccaaaaaacatccctatccctaagaaggttatgatataaaatgcccacaagaaatctatgtctgctttaatctgtcttttattgctttggaaggatggctattacatttttagtttttgctgtgaatacctgagcagtttctctcatccatacttatccttcacacatcagaagtcaggatagaatatgaatcattttaaaaacttttacaactccagagccatgtgcataagaagcattcaaaacttgccaaaacatacattttttttcaaatttaaagatactctatttttgtattcaatagctcaacaactgtggtccccactgataaagtgaagtggacaaggagacaagtaatggcataagtttgtttttcccaaagtatgcctgttcaatagccattggatgtgggaaatttctacatctcttaaaattttacagaaaatacatagccagatagtctagcaaaagttcaccaagtcctaaattgcttatccttacttcactaagtcatgaaatcattttaatgaaaagaacatcacctaggttttgtggtttctttttttcttattcatggctgagtgaaaacaacaatctctgtttctccctagcatctgtggactatttaatgtaccattattccacactctatggtccttactaaatacaaaattgaacaaaaagcagtaaaacaactgactcttcacccatattataaaatataatccaagccagattagtcaacatccataagatgaatccaagctgaactgggcctagattattgagttcaggttggatcacatccctatttattaataaacttaggaaagaaggccttacagaccatcagttagctggagctaatagaacctacacttctaaagttcggcctagaatcaatgtggccttaaaagctgaaaagaagcaggaaagaacagttttcttcaataatttgtccaccctgtcactggagaaaatttaagaatttgggggtgttggtagtaagttaaacacagcagctgttcatggcagaaattattcaatacataccttctctgaatatcctataaccaaagcaaagaaaaacaccaaggggtttgttctcctccttggagttgacctcattccaaggcagagctcaggtcacaggcacaggggctgcgcccaagcttgtccgcagccttatgcagctgtggagtctggaagactgttgcaggactgctggcctagtcccagaatgtcagcctcattttcgatttactggctcttgttgctgtatgtcatgctgaccttattgttaaacacaggtttgtttgctttttttccactcatggagacatgggagaggcattatttttaagctggttgaaagctttaaccgataaagcatttttagagaaatgtgaatcaggcagctaagaaagcatactctgtccattacggtaaagaaaatgcacagattattaactctgcagtgtggcattagtgtcctggtcaatattcggatagatatgaataaaatatttaaatggtattgtaaatagttttcaggacatatgctatagcttatttttattatcttttgaaattgctcttaatacatcaaatcctgatgtattcaatttatcagatataaattattctaaatgaagcccagttaaatgtttttgtcttgtcagttatatgttaagtttctgatctctttgtctatgacgtttactaatctgcatttttactgttatgaattattttagacagcagtggtttcaagctttttgccactaaaaataccttttattttctcctcccccagaaaagtctataccttgaagtatctatccaccaaactgtacttctattaagaaatagttattgtgttttcttaatgttttgttattcaaagacatatcaatgaaagctgctgagcagcatgaataacaattatatccacacagatttgatatattttgtgcagccttaacttgatagtataaaatgtcattgctttttaaataatagttagtcaatggacttctatcatagctttcctaaactaggttaagatccagagctttggggtcataatatattacatacaattaagttatctttttctaagggctttaaaattcatgagaataaccaaaaaaggtatgtggagagttaatacaaacataccatattcttgttgaaacagagatgtggctctgcttgttctccataaggtagaaatactttccagaatttgcctaaactagtaagccctgaatttgctatgattagggataggaagagattttcacatggcagactttagaattcttcactttagccagtaaagtatctccttttgatcttagtattctgtgtattttaacttttctgagttgtgcatgtttataagaaaaatcagcacaaagggtttaagttaaagcctttttactgaaatttgaaagaaacagaagaaaatatcaaagttctttgtattttgagaggattaaatatgatttacaaaagttacatggagggctctctaaaacattaaattaattattttttgttgaaaagtcttactttaggcatcattttattcctcagcaactagctgtgaagcctttactgtgctgtatgccagtcactctgctagattgtggagattaccagtgttcccgtcttctccgagcttagagttggatggggaataaagacaggtaaacagatagctacaatattgtactgtgaatgcttatgctggaggaagtacagggaactattggagcacctaagaggagcacctaccttgaatttaggggttagcagaggcatcctgaaaaaagtcaaagctaagccacaatctataagcagtttaggaattagcagaacgtgcgtggtgaggagatgccaaaggcaagaagagaagagtattccaaacaggagggattccaaagagagaagagtatcccaaacaacatttgcacaaacctgatggggagagagaatgtggggtggggatggatgatgagactgaagaagaaagccaggtctagataatcagtggccttgtacaccatgttaaagagtgtagacttgattctgttgtaaacaggaaagcagcacaattcatatgaatattttagaagactcccactggaatatggagaataaagttggagatgactaatcctggaagcagggagaacatttttgaggaagttgcactattttggtgaaaatgatgatcataaacatgaagaattgtaggtgatcatgacctcctctctaattttccagaagggttttggaagatataacataggaacattgacaggactgacgaaaggagatgaaatacaccatataaattgtcaaacacaaggccagatgtctaattattttgcttatgtgttgaaattacaaatttttcatcaggaaaccaaaaactacaaaacttagttttcccaagtcccagaattctatctgtccaaacaatctgtaccactccacctatatccctacctttgcatgtctgtccaacctcaaagtccaggtctatacacacgggtaagactagagcagttcaagtttcagaaaatgagaaagaggaactgagttgtgctgaacccatacaaaataaacacattctttgtatagattcttggaacctcgagaggaattcacctaactcataggtatttgatggtatgaatccatggctgggctcggcttttaaaaagccttatctgggattccttctatggaaccaagttccatcaaagcccatttaaaagcctacattaaaaacaaaattcttgctgcattgtatacaaataatgatgtcatgatcaaataatcagatgccattatcaagtggaattacaaaatggtatacccactccaaaaaaaaaaaaaaagctaaattctcagtagaacattgtgacttcatgagccctccacagccttggagctgaggagggagcactggtgagcagtaggttgaagagaaaacttggcgcttaataatctatccatgttttttcatctaaaagagccttctttttggattaccttattcaatttccatcaaggaaattgttagttccactaaccagacagcagctgggaaggcagaagcttactgtatgtacatggtagctgtgggaaggaggtttctttctccaggtcctcactggccatacaccagtcccttgttagttatgcctggtcatagacccccgttgctatcatctcatatttaagtctttggcttgtgaatttatctattctttcagcttcagcactgcagagtgctgggactttgctaacttccatttcttgctggcttagcacattcctcataggcccagctcttttctcatctggccctgctgtggagtcaccttgccccttcaggagagccatggcttaccactgcctgctaagcctccactcagctgccaccacactaaatccaagcttctctaagatgttgcagactttacaggcaagcataaaaggcttgatcttcctggacttccctttacttgtctgaatctcacctccttcaactttcagtctcagaatgtaggcatttgtcctctttgccctacatcttccttcttctgaatcatgaaagcctctcacttcctcttgctatgtgctggaggcttctgtcaggttttagaatgagttctcatctagtcctagtagcttttgatgcttaagtccaccttttaaggatacctttgagatttagaccatgtttttcgcttgagaaagccctaatctccagacttgcctttctgtggatttcaaagaccaactgaggaagtcaaaagctgaatgttgactttctttgaacatttccgctataacaattccaattctcctcagagcaatatgcctgcctccaactgaccaggagaaaggtccagtgccaaagagaaaaacacaaagattaattatttcagttgagcacatactttcaaagtggtttgggtattcatatgaggttttctgtcaagagggtgagactcttcatctatccatgtgtgcctgacagttctcctggcactggctggtaacagatgcaaaactgtaaaaattaagtgatcatgtattttaacgatatcatcacatacttattttctatgtaatgttttaaatttcccctaacatactttgactgttttgcacatggtagatattcacatttttttgtgttgaagttgatgcaatcttcaaagttatctaccccgttgcttattagtaaaactagtgttaatacttggcaagagatgcagggaatctttctcatgactcacgccctatttagttattaatgctactaccctattttgagtaagtagtaggtccctaagtacattgtccagagttatacttttaaagatatttagccccatatacttcttgaatctaaagtcatacaccttgctcctcatttctgagtgggaaagacatttgagagtatgttgacaattgttctgaaggtttttgccaagaaggtgaaactgtcctttcatctgtgtatgcctggggctgggtccctggcagtgatggggtgacaatgcaaagctgtaaaaactaggtgctagtgggcacctaatatcatcatcatatacttattttcaagctaatatgcaaaatcccatctctgtttttaaactaagtgtagatttcagagaaaatattttgtggttcacataagaaaacagtctactcagcttgacaagtgttttatgttaaattggctggtggtttgaaatgaatcatcttcacataatgttttctttaaaaatattgtgaatttaactctaattcttgttattctgtgtgataataaagaataaactaatttctata

SEQ ID NO: 2 - DNA, 60 bp, Artificial Sequence
  probe polynucleotide
ccacaggtttggcttttgttaaaatgtttgatatcttcgatgttgatctctgtctgcaat

SEQ ID NO: 3 - DNA, 60 bp, Artificial Sequence
  probe polynucleotide
acataagaaaacagtctactcagcttgacaagtgttttatgttaaattggctggtggttt

SEQ ID NO: 4 - DNA, 60 bp, Artificial Sequence
  probe polynucleotide
tccctggcagtgatggggtgacaatgcaaagctgtaaaaactaggtgctagtgggcacct

SEQ ID NO: 5 - DNA, 25 bp, Artificial Sequence
  forward primer
ttgtcctctaatgaggtattgcgag

SEQ ID NO: 6 - DNA, 24 bp, Artificial Sequence
  reverse primer
attgcagacagagatcaacatcga

SEQ ID NO: 7 - DNA, 21 bp, Artificial Sequence
  forward primer
tatggcttcaccaaatggaaa

SEQ ID NO: 8 - DNA, 26 bp, Artificial Sequence
  reverse primer
tgtgaaaatctcttcctatccctaat

SEQ ID NO: 9 - DNA, 27 bp, Artificial Sequence
  forward primer
atcagatgccattatcaagtggaatta

SEQ ID NO: 10 - DNA, 26 bp, Artificial Sequence
  reverse primer
ccagccaatttaacataaaacacttg

SEQ ID NO: 11 - DNA, 26 bp, Artificial Sequence
  forward primer
caaaacttcttgataacttgcatgat

SEQ ID NO: 12 - DNA, 24 bp, Artificial Sequence
  forward primer
tcgatgttgatctctgtctgcaat

SEQ ID NO: 13 - DNA, 25 bp, Artificial Sequence
  forward primer
tggttcacataagaaaacagtctac

SEQ ID NO: 14 - DNA, 18 bp, Artificial Sequence
  reverse primer
catttcaaaccaccagcc

SEQ ID NO: 15 - DNA, 26 bp, Artificial Sequence
  forward primer
aagaaaacagtctactcagcttgaca

SEQ ID NO: 16 - DNA, 25 bp, Artificial Sequence
  reverse primer
atgtgaagatgattcatttcaaacc

SEQ ID NO: 17 - DNA, 22 bp, Artificial Sequence
  labeled Taqman probe
tgttttatgttaaattggctgg

(19) United States
(12) Patent Application Publication
(10) Pub. No.: US 2005/0255481 A1
(43) Pub. Date: Nov. 17, 2005

(54) Progesterone receptor transcript sequences

(76) Inventors: Xiao-Jun Ma, San Diego, CA (US); Edward Enright, San Diego, CA (US); Mark G. Erlander, Encinitas, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(21) Appl. No.: 10/843,717

(22) Filed: May 11, 2004

(51) Int. Cl. C12Q 1/00; C12Q 1/68; C07H 21/04; C12N 15/09; C07K 14/72

(52) U.S. Cl. 435/6; 435/69.1; 435/320.1; 435/325; 530/350; 536/23.5

(57) ABSTRACT

The invention relates to the identification and use of sequences from expressed progesterone receptor transcripts in relation to breast cancer. In particular, the invention provides the identities of polynucleotide sequences that may be used to identify populations that are positive for estrogen receptor expression. The expressed polynucleotide sequences may be used in the study and/or diagnosis of cells and tissue in breast cancer as well as for the study and/or determination of prognosis of a patient.

### FIELD OF THE INVENTION

The invention relates to the identification and use of sequences from expressed progesterone receptor transcripts in relation to cancer, including breast cancer. In particular, the invention provides the identities of polynucleotide sequences that may be used to identify populations that are positive for progesterone receptor expression, which may also be correlated with estrogen receptor expression, in both normal and tumor cells. The expressed polynucleotide sequences may be used in the study and/or diagnosis of cells and tissue in cancer as well as for the study and/or determination of prognosis of a patient.

### BACKGROUND OF THE INVENTION

Major and intensive research has been focused on early detection, treatment and prevention of cancer. With respect to breast cancer, this has included an emphasis on determining the presence of precancerous or cancerous ductal epithelial cells. These cells are analyzed, for example, for cell morphology, for protein markers, for nucleic acid markers, for chromosomal abnormalities, for biochemical markers, and for other characteristic changes that would signal the presence of cancerous or precancerous cells. This has led to various molecular alterations that have been reported in breast cancer, few of which have been well characterized in human clinical breast specimens. Molecular alterations include presence/absence of estrogen and progesterone steroid receptors, HER-2 expression/amplification (Mark H F, et al., “HER-2/neu gene amplification in stages I-IV breast cancer detected by fluorescent in situ hybridization.” Genet Med; 1(3):98-103 1999), Ki-67 (an antigen that is present in all stages of the cell cycle except GO and used as a marker for tumor cell proliferation, and prognostic markers (including oncogenes, tumor suppressor genes, and angiogenesis markers) like p53, p27, Cathepsin D, pS2, multi-drug resistance (MDR) gene, and CD31.

Estrogen receptor (ER) status has been of particular interest because it has been correlated with prognosis and treatment regimens. Generally speaking, patients identified as having ER positive breast cancer biopsies have a better overall survival expectation while patients with ER negative biopsies are treated more aggressively, such as with immediate chemotherapy after surgical intervention, because of a poor prognosis. The status of progesterone receptor (PR) expression in breast cancer has also been of particular interest.

The human PR gene sequence is identified by UniGene cluster Hs.2905, with 12 deposited polynucleotide sequences identified as messenger RNA (mRNA) belonging to the cluster. The 12 sequences contain either complete or partial coding regions for the PR protein. The accession numbers in GenBank for the sequences are as follows: AF016381.1, AY212933.1, NM_{—}000926.2, AY382152.1, AY382151.1, M15716.1, AB084248.1, AB085683.1, AB085845.1, AB085844.1, AB085843.1, and X51730.1. None of the sequences, however, are identified as having a polyadenylation signal.

Citation of documents herein is not intended as an admission that any is pertinent prior art. All statements as to the date or representation as to the contents of documents is based on the information available to the applicant and does not constitute any admission as to the correctness of the dates or contents of the documents.

### SUMMARY OF THE INVENTION

The present invention relates to the identification and use of polynucleotide sequences which are expressed as part of human progesterone receptor (PR) transcripts. These sequences were not previously identified as being expressed in association with the PR gene. Like known PR transcripts, the expression of the sequences of the invention correlate with (and is thus able to identify) cells that are positive for PR expression, which may be correlated with estrogen receptor (ER) expression (usually expression of the ESR1, or estrogen receptor alpha, encoded protein), in breast cancer specimens.

PR expression has been associated with multiple tumor types beyond those of the breast and has been implicated in other medical conditions. See for example, Marosi, C. et al. “Guidelines to the treatment of meningioma.” Forum (Genova), 2003, 13(1):76-89; Bodner K. et al., “Estrogen and progesterone receptor expression in patients with uterine smooth muscle tumors.” Fertil Steril. 2004, 81(4):1062-6; Arnett-Mansfield R. L. et al., “Subnuclear distribution of progesterone receptors A and B in normal and malignant endometrium.” J Clin Endocrinol Metab. 2004, 89(3): 1429-42; Remoue F. et al., “High intraepithelial expression of estrogen and progesterone receptors in the transformation zone of the uterine cervix.” Am J Obstet Gynecol. 2003, 189(6):1660-5; Sasaki M. et al., “Methylation and inactivation of estrogen, progesterone, and androgen receptors in prostate cancer.” J Natl Cancer Inst. 2002, 94(5):384-90; Lee W. Y. et al., “Papillary cystic tumors of the pancreas: assessment of malignant potential by analysis of progesterone receptor, flow cytometry, and ras oncogene mutation.” Anticancer Res. 1997, 17(4A):2587-91; Lewy-Trenda I. et al., “Estrogen and progesterone receptors in neoplastic and non-neoplastic thyroid lesions.” Pol J Pathol. 2002, 53(2):67-72; Kuebler J. F. et al., “Progesterone administration after trauma and hemorrhagic shock improves cardiovascular responses.” Crit Care Med. 2003, 31(6):1786-93; Kuebler J. F. et al., “Administration of progesterone after trauma and hemorrhagic shock prevents hepatocellular injury.” Arch Surg. 2003, 138(7):727-34.

The invention thus provides for detecting expression of the disclosed PR transcript sequences in correlation with meningioma, smooth muscle tumors (such as those of the uterus), tumors of the endometrium or cervix, prostate tumors, pancreatic tumors, and thyroid tumors as well as in correlation with any condition affecting an organism or tissue type wherein PR expression is increased or decreased relative to normal cells of the same tissue type that are not afflicted with the condition. The invention also provides for the use of the disclosed PR transcript sequences in determining PR expression, whether at normal, increased, or decreased levels, in any tissue type. Non-limiting examples of the latter include the detection of PR expression in cardiac tissues, such as those of the left ventricle, and liver tissues, including hepatocytes at injury sites.

The expressed polynucleotide sequences are identified herein as belonging to the 3′ untranslated region (UTR) of the human progesterone receptor (PR) gene, although the invention does not rely on this identification for its practice. The invention is based in part on the expression of these sequences, independent of whether they are expressed as all or part of the 3′ UTR of the PR gene, in cells in correlation with PR or ER positive status. Thus the invention includes the ability to assay cancer cells, including breast cancer cells, for PR expression by use of the disclosed sequences. The sequences may thus serve as a supplement to assays for ER status in breast cancer samples or used as a substitute for known assays for PR and/or ER status. The expression of the sequences may also be used in diagnostic or prognostic methods or assays in the clinic to determine the course of treatment following identification of the presence of breast cancer or subsequent surgical removal thereof.

The present invention provides a non-subjective means for the identification of PR and/or ER status in breast cancer samples by assaying for the expression of the disclosed sequences. Thus subjective interpretation is less necessary, and a more accurate assessment of PR and/or ER status, and breast cancer status and prognosis, is provided. Furthermore, the expression of the disclosed sequences can also be used as a means to assay small, node negative tumors that are not readily assayed by other means.

The expressed polynucleotide sequences of the invention comprise sequences which were not previously identified as being expressed in association with expression of PR or ER. These include sequences associated with two UniGene Clusters, Hs.32405 and Hs. 154918, that were not previously associated with each other or with the PR cluster (Hs.2905). The invention provides a lengthened PR transcript (coding region included) sequence identified as SEQ ID NO:1, which combines sequences from X51730.1 (positions 1 to 5003, inclusive) with sequences identified by the instant invention (positions 5004 to 13753, the last position shown in SEQ ID NO:1). While no polyadenylate (polyA) tail is shown as part of SEQ ID NO:1, the invention includes the addition of a polyA tail after any one of positions 13746 to 13753, inclusive. Preferred positions for the start of a polyA tail are after position 13746 or 13750 of SEQ ID NO:1. Therefore, the invention includes 3′ UTR sequences ending at any one of positions 13746 to 13753, inclusive. The invention also provides for an isolated polynucleotide comprising the human progesterone receptor 3′ UTR, which starts at the termination codon at the end of the PR gene and comprises at least 1-2 nucleotides of the polyA tail.

While expression of the transcribed portion of SEQ ID NO:1, in whole or in part, may be used in the practice of the invention, the invention includes the discovery that sequences within the 3′ end of SEQ ID NO:1 are better correlated with ER expression than sequences within the PR coding region. The invention thus provides for the use of all or part of the sequence from positions 5004 to 13753 of SEQ ID NO:1, inclusive, and with possible addition of and thus adjustment for polyA tails as described above, as correlated with PR and/or ER expression. These sequences may thus be used in relation to cancer, including breast cancer, prognosis and treatment. These sequences may also be used more generally to detect PR expression in non-cancerous cells as well as cells under various conditions.

Positions 5004 to 13753 of SEQ ID NO:1 can also be viewed as being made up of four different regions. The first region is 244 nucleotides long and is from positions 5004 to 5247 of SEQ ID NO:1, inclusive, which contains sequences not previously associated with any transcript or gene cluster.

The second region is 2648 nucleotides long and is from positions 5248 to 7895 of SEQ ID NO:1, inclusive, which contains sequences found in UniGene cluster Hs.154918. The third region is 491 nucleotides long and is from positions 7896 to 8386 of SEQ ID NO:1, inclusive, which contains sequences not previously associated with any transcript or gene cluster. The fourth region is 5367 nucleotides long and is from 8387 to 13753 of SEQ ID NO:1, inclusive, which contains sequences found in UniGene cluster Hs.32405 as well as the starting positions of a polyA tail as described above. Partial sequences from within any of these regions may be used in the practice of the invention, with sequences from within the first, third and fourth regions being preferred. Of course sequences that overlap two or more of these regions may also be used in the practice of the invention. Additionally, the invention provides for the use of fragments and homologs of the disclosed sequences as described herein.

The expression of the sequences of the invention are capable of discriminating between cancer cells, such as breast cancer cells, that are PR positive or PR negative with significant accuracy. The PR expression level may also be correlated with ER expression status in some cases, such as those of breast cancer cells. The sequences are identified as correlated with PR and/or ER expression status in breast cancer such that the levels of their expression are relevant to a determination of PR and/or ER status in a breast cancer cell. Thus in one aspect, the invention provides a method to determine the PR and/or ER status of breast cancer of a subject afflicted with, or suspected of having, breast cancer by assaying a cell containing sample from said subject for expression of one or more than one sequence disclosed herein. The expression of sequences that are highly correlated with PR and/or ER status may be detected and used to assay an sample from a subject afflicted with, or suspected of having, breast cancer to identify the PR and/or ER status of breast cancer to which the sample belongs. The detection of PR expression may be done in combination with, or separate from, a direct assay for ER expression. Such assays may be used as part of a method to determine the therapeutic treatment for said subject based upon the PR and/or ER status identified.

The invention may advantageously be used to identify a subject's breast cancer sample as being either ER positive, where the subject may be treated based upon ER positive status, or ER negative, where other therapeutic treatments may be used on the subject to reduce the time spent with treatments based upon ER positive status (and thus avoid time loss in the event that such treatments lack efficacy). The present invention also provides for the advantageous ability to determine PR and/or ER status in combination with other information to provide more detailed information in diagnosing and treating breast cancer.

The disclosed sequences may be used singly with significant accuracy or in combinations of two or more. The present invention thus provides means for correlating a molecular expression phenotype with PR and/or ER expression and thus a physiological (cellular) state. This correlation also provides a way to molecularly diagnose and/or monitor a cell's status. Additional uses of the correlated sequences are in the classification of cells and tissues; determination of diagnosis and/or prognosis; and determination and/or alteration of therapy.

An assay of the invention may utilize a means related to the expression level of the sequences disclosed herein as long as the assay reflects, quantitatively or qualitatively, expression of the sequence. Preferably, however, a quantitative assay means is preferred. The ability to discriminate is conferred by the identification of expression of the individual sequences as relevant and not by the form of the assay used to determine the actual level of expression. An assay of the invention may utilize any identifying feature of a sequence as disclosed herein as long as the assay reflects, quantitatively or qualitatively, expression of the sequence, optionally independent of actual expression of PR protein. An assay may simply be to detect the expression of a nucleic acid molecule that contains any sequence, or portion thereof, disclosed herein in correlation with cancer, including breast cancer.

Identifying features include, but are not limited to, unique nucleic acid sequence portions from within the disclosed sequences. Means for the practice of the invention include detection of nucleic acid amplification as indicative of increased expression levels and nucleic acid inactivation, deletion, or methylation, as indicative of decreased expression levels. Stated differently, the invention may be practiced by assaying one or more aspect of the DNA template underlying the expression of the disclosed sequences, or of the RNA used as an intermediate to express the sequences. As such, the detection of the presence of, amount of, stability of, or degradation (including rate) of, such DNA or RNA molecules may be used in the practice of the invention. As such, the invention may be practiced with one or more of the disclosed sequences necessary to discriminate between PR positive and negative samples and an appropriate cell containing sample for use in an expression assay.

In one aspect, the invention provides for the identification of sequence expression alone or as part of analyzing global, or near global, gene expression from single cells or homogenous cell populations which have been dissected away from, or otherwise isolated or purified from, contaminating cells beyond that possible by a simple biopsy. In a further aspect, one or more sequences capable of discriminating between PR and/or ER positive and negative samples may be used to identify PR and/or ER status of an unknown sample of cell(s), such as those from the breast. Preferably, the sample is isolated via non-invasive means. The expression of said sequence(s) in said unknown sample may be determined and compared to the expression of said sequence(s) in reference data of gene expression patterns from PR and/or ER positive and/or negative samples. Alternatively, the expression level may be compared to expression levels in normal or non-cancerous cells, preferably from the same sample or subject.

In embodiments of the invention utilizing quantitative PCR to detect expression of the sequences disclosed herein, the expression level may be compared to expression levels of reference genes in the same sample, such as, but not limited to, by use of a ratio of the expression levels of a disclosed sequence and a reference gene. The use of a ratio can reduce the need for comparisons with normal or non-cancerous cells.

The invention may be practiced with cell containing samples wherein contaminating, non-breast cells (such as infiltrating lymphocytes or other immune system cells) have been removed by dissection (such as laser capture microdissection or analogous microdissection methods) to remove or reduce their possible effects on identifying the expression status of the disclosed sequences in cancer cells, including suspected breast cancer cells. Such contamination may be present where a biopsy is used directly to detect the expression status of various sequences.

While the present invention has been described mainly in the context of human breast cancer, it may be practiced in the context of other human cancers as well as cancer of any animal known to be potentially afflicted by cancer by use of sequences disclosed herein which are sufficiently homologous to also be useful for the detection of cancer in other animals. Preferred animals for the application of the present invention are mammals, particularly those important to agricultural applications (such as, but not limited to, cattle, sheep, horses, and other “farm animals”) and for human companionship (such as, but not limited to, dogs and cats).

### BRIEF DESCRIPTION OF THE FIGURES

FIG. 1 shows a representation of the PR-UTR region showing select PCR products and locations of 60 mer oligonucleotides in regions 2 (“R2”) and 4 (“R4”) of the disclosed 3′UTR.

FIG. 2 shows a physical link between the known PR transcript and region 2 as demonstrated by PCR.

FIG. 3 shows a physical link between regions 2 and 4 of the disclosed 3′ UTR.

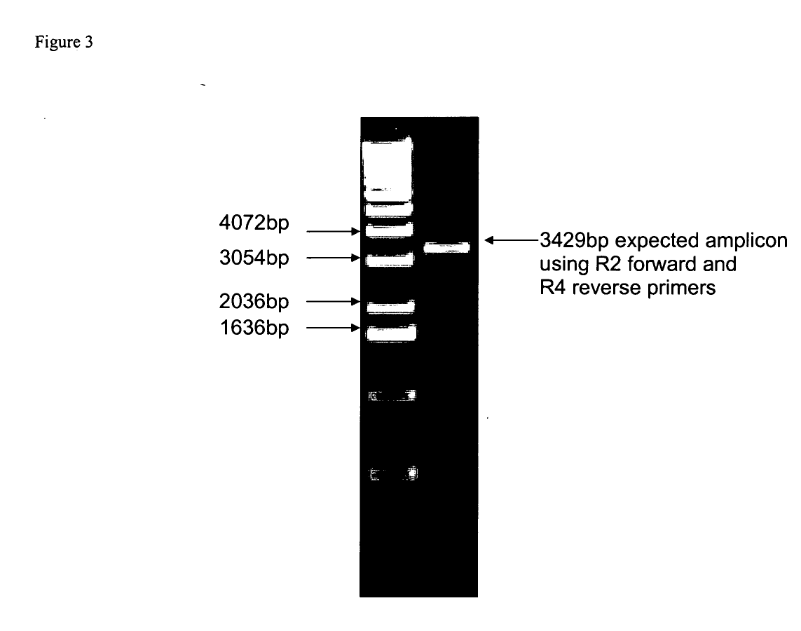

FIG. 4 shows the results of a digestion with restriction enzymes which confirms the identity of the PCR product shown in FIG. 3.

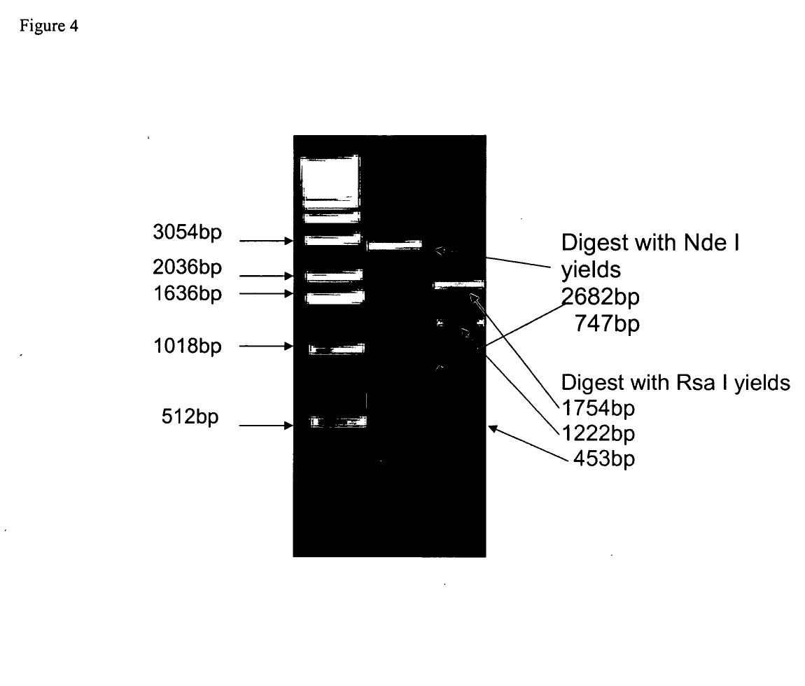

FIG. 5 shows a representation of the long PCR products between known PR gene sequences and the 60 mer oligonucleotide in region 4.

FIG. 6 shows four long PCR amplification reactions confirming a physical link between the known PR transcript and region 4.

FIG. 7 shows an illustration of UniGene clusters along chromosome 11.

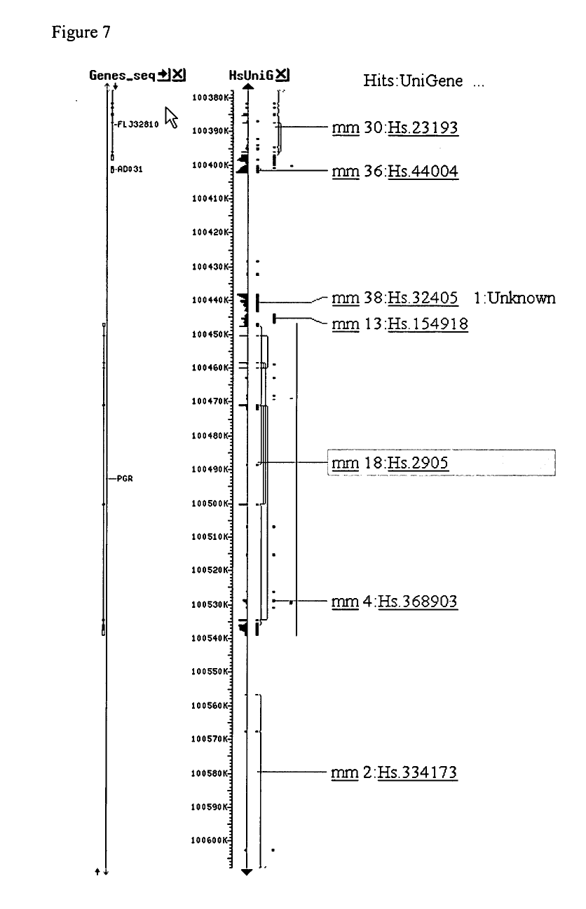

### DETAILED DESCRIPTION OF THE SPECIFIC EMBODIMENTS

In a first embodiment, the invention provides for a polynucleotide comprising a progesterone receptor (PR) cDNA comprising all or part of positions 5004 to 13753, inclusive, of SEQ ID NO:1 and optionally all or part of positions 1-5003, inclusive, of SEQ ID NO:1 or all or part of the PR coding region. The invention thus provides for a polynucleotide comprising a full length cDNA (SEQ ID NO:1) as well as partial cDNAs comprising the 3′ UTR, such as positions 5004 to 13753, inclusive, of SEQ ID NO:1. Of course polynucleotides consisting of SEQ ID NO:1 or positions 5004 to 13753, inclusive, of SEQ ID NO:1 are also provided by the invention.

Also provided by the invention are polynucleotides comprising all or part of positions 5004 to 13753, inclusive, of SEQ ID NO:1 and at least 154 contiguous nucleotides of the PR coding region, wherein coding region does not include the termination codon. While PR cDNAs comprising even one nucleotide from positions 5004 to 13753, inclusive, of SEQ ID NO:1 are previously unknown, preferred embodiments of the invention include those with various lengths of sequence, as discussed below, from positions 5004 to 13753, inclusive, of SEQ ID NO:1.

The invention also provides for primer and probe polynucleotides of about 15 to about 900 nucleotides in length for the detection of the PR expression and/or expression of 3′ UTR sequences as disclosed herein. Such polynucleotides may comprise about 15 to about 500 (but less than 550) contiguous nucleotides of positions 5004 to 13753, inclusive, of SEQ ID NO:1, which may be used in combination with primers that are complementary, in whole or in part, to known PR mRNA sequences. Primers and probes of the invention may also be complementary, in whole or in part, to all or part of the four regions in positions 5004 to 13753 as described herein.

Preferred embodiments of such primers are those of about 15 to about 60 nucleotides in length and comprising from about 15 to 60 contiguous nucleotides from known PR mRNA sequences or from positions 5004 to 13753, inclusive, of SEQ ID NO:1. Non-limiting examples include those provided in the Examples herein.

Preferred probe polynucleotides of the invention may be of various sizes, such as about 60 nucleotides or more in length. Non-limiting examples of such probes include polynucleotides comprising CCACAGGTTTGGCTTTTGTTAAAATGTTTGATATCTTCGATGTTGATCTCTGTCTG CAAT (SEQ ID NO:2), which hybridizes to region 2 of the disclosed 3′ UTR sequence or ACATAAGAAAACAGTCTACTCAGCTTGACAAGTGTTTTATGTTAAATTGGCTGGT GGTTT (SEQ ID NO:3), which hybridizes to region 4 of the disclosed 3′ UTR sequence.

In some embodiments of the invention, however, the polynucleotide is not a 60 mer consisting of TCCCTGGCAGTGATGGGGTGACAATGCAAAGCTGTAAAAACTAGGTGCTAGTGG GCACCT (SEQ ID NO:4), which is disclosed in WO02/10449.

In another embodiment, the invention provides isolated nucleic acid molecules which can be used to detect expression of the 3′ UTR of human PR. Non-limiting examples of such molecules include those which hybridize to the 3′ UTR of human PR under stringent conditions. Preferably, the nucleic acid molecules are of about the same length or shorter than that of the 3′ UTR sequence being hybridized to or of a length from about 15 to about 8900 nucleotides. In some embodiments of the invention, the molecule has a length like those of the primers and probes described herein.

Other isolated nucleic acid molecules include those having a length from about 15 to about 8900 nucleotides which are substantially identical to the 3′ UTR sequences disclosed herein. Such molecules are at least about 85% identical, at least about 90%, at least about 95%, at least about 96%, at least about 97%, at least about 98%, or at least about 99% identical to the 3′ UTR of human PR.

In a further embodiment, the invention provides methods of quantitative PCR (Q-PCR) analysis comprising quantitative PCR amplification of all or part of positions 5004 to 13753, inclusive, of SEQ ID NO:1. Of course appropriate primers for the performance of such analysis by Q-PCR may be readily designed by reference to the sequences disclosed herein. The primers include those which are complementary, in whole or in part, to PR sequences upstream (5′ of) positions 5004 to 13753, inclusive, of SEQ ID NO:1 as well as primers that are complementary, in whole or in part, to the polyA tail of PR transcripts.

The use of Q-PCR preferably is by amplification of at least 50 nucleotides, at least 75 nucleotides, at least 100 nucleotides, at least 125 nucleotides, at least 150 nucleotides, at least 175 nucleotides, at least 200 nucleotides, at least 225 nucleotides, at least 250 nucleotides, at least 275 nucleotides, or at least 300 nucleotides of positions 5004 to 13753, inclusive, of SEQ ID NO:1. The amplification of more than 300 nucleotides is also within the scope of the invention. The use of Q-PCR analysis is preferably practiced on the nucleic acids of a biological sample, such as that obtained from a cancer patient.

Additional embodiments of the invention include methods of preparing a polynucleotide containing all or part of the 3′ untranslated region of a progesterone receptor transcript, said method comprising PCR amplification using a first primer which hybridizes to all or part of positions 1 to 5003, inclusive, of SEQ ID NO:1 and a second primer which hybridizes to a sequence from within positions 5004 to 13753, inclusive, of SEQ ID NO:1 or which hybridizes to the region comprising the polyA tail of a PR transcript. In some embodiments, the first primer hybridizes to all or part of the PR coding region. In other embodiments, the methods comprising PCR amplification using a pair of primers which hybridize to all or part of positions 5004 to 13753, inclusive, of SEQ ID NO:1. Of course nucleic acid molecules comprising a polynucleotide prepared by such methods are within the scope of the invention.

Yet another embodiment of the invention provides methods of detecting the expression of human PR. Some methods comprise obtaining a nucleic acid containing sample from a human subject; and amplifying all or part of positions 5004 to 13753, inclusive, of SEQ ID NO:1 by quantitative PCR wherein detection of the amplified sequence is indicative of PR expression. Other methods comprise obtaining a nucleic acid containing sample from a human subject; and detecting hybridization between a probe comprising at least 15 contiguous nucleotides of positions 5004 to 13753, inclusive, of SEQ ID NO:1 to the nucleic acids of said sample, wherein either said sample or said probe is labeled with a detectable marker.

Before continuing with additional embodiments of the invention, a few definitions are provided to aid in the understanding of the invention.

A “gene” is a polynucleotide that encodes a discrete product, whether RNA or proteinaceous in nature. It is appreciated that more than one polynucleotide, such as, but not limited to, alternatively spliced mRNA molecules, may be capable of encoding a discrete product. The term includes alleles and polymorphisms of a gene that encodes the same product, or a functionally associated (including gain, loss, or modulation of function) analog thereof, based upon chromosomal location and ability to recombine during normal mitosis.

A “sequence” or “gene sequence” as used herein is a nucleic acid molecule or polynucleotide composed of a discrete order of nucleotide bases. The term includes the ordering of bases that encodes a discrete product (i.e. “coding region”), whether RNA or proteinaceous in nature, as well as the ordered bases that precede or follow a “coding region”, and fragments of such contiguous base sequences. Non-limiting examples include the 3′ untranslated regions (UTRs) of the PR gene and fragments and homologs thereof. It is appreciated that alleles and polymorphisms of the disclosed 3′ UTR sequences may exist and may be used in the practice of the invention to identify the expression level(s) of the disclosed sequences or the allele or polymorphism. Identification of an allele or polymorphism depends in part upon chromosomal location and ability to recombine during mitosis.

The terms “correlate” or “correlation” or equivalents thereof refer to an association between expression of one or more sequences and the ER status of a breast cancer cell and/or a breast cancer patient. A sequence of the invention is expressed at higher levels in correlation with ER positive status and thus the corresponding breast cancer survival or outcome. Increases may be readily expressed in the form of a ratio between expression in a non-normal cell and a normal cell such that a ratio of one (1) indicates no difference while ratios of two (2) and one-half indicate twice as much, and half as much, expression in the non-normal cell versus the normal cell, respectively. Expression levels can be readily determined by quantitative methods as described below.

For example, increases in gene expression can be indicated by ratios of or about 1.1, of or about 1.2, of or about 1.3, of or about 1.4, of or about 1.5, of or about 1.6, of or about 1.7, of or about 1.8, of or about 1.9, of or about 2, of or about 2.5, of or about 3, of or about 3.5, of or about 4, of or about 4.5, of or about 5, of or about 5.5, of or about 6, of or about 6.5, of or about 7, of or about 7.5, of or about 8, of or about 8.5, of or about 9, of or about 9.5, of or about 10, of or about 15, of or about 20, of or about 30, of or about 40, of or about 50, of or about 60, of or about 70, of or about 80, of or about 90, of or about 100, of or about 150, of or about 200, of or about 300, of or about 400, of or about 500, of or about 600, of or about 700, of or about 800, of or about 900, or of or about 1000. A ratio of 2 is a 100% (or a two-fold) increase in expression.

A “polynucleotide” is a polymeric form of nucleotides of any length, either ribonucleotides or deoxyribonucleotides. This term refers only to the primary structure of the molecule. Thus, this term includes double- and single-stranded DNA and RNA. It also includes known types of modifications including labels known in the art, methylation, “caps”, substitution of one or more of the naturally occurring nucleotides with an analog, and internucleotide modifications such as uncharged linkages (e.g., phosphorothioates, phosphorodithioates, etc.), as well as unmodified forms of the polynucleotide. Preferred polynucleotides are those containing sequences that are isolated from other sequences with which they are found in nature. Recombinant polynucleotides that are a combination of sequences not normally found in nature are also within the scope of the invention.

The term “amplify” is used in the broad sense to mean creating an amplification product can be made enzymatically with DNA or RNA polymerases. “Amplification,” as used herein, generally refers to the process of producing multiple copies of a desired sequence, particularly those of a sample. “Multiple copies” mean at least 2 copies. A “copy” does not necessarily mean perfect sequence complementarity or identity to the template sequence.

Methods for amplifying mRNA are generally known in the art, and include reverse transcription PCR (RT-PCR) and those described in U.S. patent application Ser. No. 10/062,857 (filed on Oct. 25, 2001), as well as U.S. Provisional Patent Applications 60/298,847 (filed Jun. 15, 2001) and 60/257,801 (filed Dec. 22, 2000), all of which are hereby incorporated by reference in their entireties as if fully set forth. Another method which may be used is quantitative PCR (or Q-PCR). Alternatively, RNA may be directly labeled as the corresponding cDNA (“complementary DNA” obtained by reverse transcription of the corresponding mRNA, optionally with subsequent replication of the cDNA) by methods known in the art. By corresponding is meant that a nucleic acid molecule shares a substantial amount of sequence identity with another nucleic acid molecule. Substantial amount means at least 95%, usually at least 98% and more usually at least 99%, and sequence identity is determined using the BLAST algorithm, as described in Altschul et al. (1990), J. Mol. Biol. 215:403-410 (using the published default setting, i.e. parameters w=4, t=17).

Expression of the disclosed sequence may be detected by use of a microarray comprising a probe which hybridizes to one or more of the disclosed sequences. A “microarray” is a linear or two-dimensional array of preferably discrete regions, each having a defined area, formed on the surface of a solid support such as, but not limited to, glass, plastic, or synthetic membrane. The density of the discrete regions on a microarray is determined by the total numbers of immobilized polynucleotides to be detected on the surface of a single solid phase support, preferably at least about 50/cm², more preferably at least about 100/cm², even more preferably at least about 500/cm², but preferably below about 1,000/cm². In some embodiments, the arrays contain less than about 500, about 1000, about 1500, about 2000, about 2500, or about 3000 immobilized polynucleotides in total. In other embodiments, the arrays can contain more than about 500, about 1000, about 1500, about 2000, about 2500, or about 3000 immobilized polynucleotides in total.

As used herein, a DNA microarray is an array of oligonucleotides or polynucleotides placed on a chip or other surfaces used to hybridize to amplified or cloned polynucleotides from a sample. If the position of one or more probes in the array is a sequence as disclosed herein, the presence, or expression, of the sequences disclosed herein in a sample of polynucleotides can be determined based on their binding to the position(s) in the microarray containing such probes.

Because the invention relies upon the use of PR 3′ UTR sequences that are expressed, one embodiment of the invention involves determining expression by hybridization of mRNA, or an amplified or cloned version thereof, of a sample cell to a polynucleotide that is unique to a particular gene sequence. Preferred polynucleotides of this type include primers and probes as described herein and contain at least about 15, at least about 17, at least about 19, at least about 20, at least about 22, at least about 24, at least about 26, at least about 28, at least about 30, or at least about 32 consecutive basepairs of a gene sequence that is not found in other gene sequences. The term “about” as used in the previous sentence refers to an increase or decrease of 1 from the stated numerical value.

Even more preferred are polynucleotides of at least or about 50, at least or about 100, at least about or 150, at least or about 200, at least or about 250, at least or about 300, at least or about 350, or at least or about 400 basepairs of a gene sequence that is not found in other gene sequences. The term “about” as used in the preceding sentence refers to an increase or decrease of 25 from the stated numerical value. Such polynucleotides may also be referred to as polynucleotide probes that are capable of hybridizing to sequences of the genes, or unique portions thereof, described herein. Preferably, the sequences are those of 3′ UTR regions encoded by the genes, the corresponding cDNA comprising such regions, and/or amplified versions of such sequences. In preferred embodiments of the invention, the polynucleotide probes are immobilized on an array, other devices, or in individual spots that localize the probes.

In another embodiment of the invention, all or part of a disclosed sequences may be amplified and detected by methods such as the polymerase chain reaction (PCR) and variations thereof, such as, but not limited to, quantitative PCR (Q-PCR), reverse transcription PCR (RT-PCR), and real-time PCR (including as a means of measuring the initial amounts of mRNA copies for each sequence in a sample), optionally real-time RT-PCR or real-time Q-PCR. Such methods would utilize one or two primers that are complementary to all or part of a disclosed sequence, such as positions 5004 to 13753, inclusive, of SEQ ID NO:1, where the primers are used to prime nucleic acid synthesis. The newly synthesized nucleic acids are optionally labeled and may be detected directly or by hybridization to a polynucleotide of the invention. The newly synthesized nucleic acids may be contacted with polynucleotides (containing sequences) of the invention under conditions which allow for their hybridization. Additional methods to detect the expression of expressed nucleic acids include RNAse protection assays, including liquid phase hybridizations, and in situ hybridization of cells.

The term “label” or “detectable marker” refer to a composition capable of producing a detectable signal indicative of the presence of the labeled molecule. Suitable labels include radioisotopes, nucleotide chromophores, enzymes, substrates, fluorescent molecules, chemiluminescent moieties, magnetic particles, bioluminescent moieties, and the like, including labels suitable for indirect detection, such as biotin. As such, a label is any composition detectable by spectroscopic, photochemical, biochemical, immunochemical, electrical, optical or chemical means.

The term “support” refers to conventional supports such as beads, particles, dipsticks, fibers, filters, membranes and silane or silicate supports such as glass slides.

As used herein, a “breast tissue sample” or “breast cell sample” refers to a sample of breast tissue or fluid isolated from an individual suspected of being afflicted with, or at risk of developing, breast cancer. Such samples are primary isolates (in contrast to cultured cells) and may be collected by any non-invasive means, including, but not limited to, ductal lavage, fine needle aspiration, needle biopsy, the devices and methods described in U.S. Pat. No. 6,328,709, or any other suitable means recognized in the art. Alternatively, the “sample” may be collected by an invasive method, including, but not limited to, surgical biopsy. A sample of the invention may also be one that has been formalin fixed and paraffin embedded (FFPE) or simply frozen after collection. The invention provides for the detection of the expression of the disclosed PR sequences in such samples.

“Expression” and “gene expression” include transcription and/or translation of nucleic acid material.

As used herein, the term “comprising” and its cognates are used in their inclusive sense; that is, equivalent to the term “including” and its corresponding cognates.

Conditions that “allow” an event to occur or conditions that are “suitable” for an event to occur, such as hybridization, strand extension, and the like, or “suitable” conditions are conditions that do not prevent such events from occurring. Thus, these conditions permit, enhance, facilitate, and/or are conducive to the event. Such conditions, known in the art and described herein, depend upon, for example, the nature of the nucleotide sequence, temperature, and buffer conditions. These conditions also depend on what event is desired, such as hybridization, cleavage, strand extension or transcription.

“Detection” includes any means of detecting, including direct and indirect detection of gene expression and changes therein. For example, “detectably less” products may be observed directly or indirectly, and the term indicates any reduction (including the absence of detectable signal). Similarly, “detectably more” product means any increase, whether observed directly or indirectly.

Increases in expression of the disclosed sequences are defined in the following terms based upon percent or fold changes over expression in normal cells. Increases may be of 10, 20, 30, 40, 50, 60, 70, 80, 90, 100, 120, 140, 160, 180, or 200% relative to expression levels in normal cells. Alternatively, fold increases may be of 1, 1.5, 2, 2.5, 3, 3.5, 4, 4.5, 5, 5.5, 6, 6.5, 7, 7.5, 8, 8.5, 9, 9.5, or 10 fold over expression levels in normal cells.

The terms “identical” or percent “identity,” in the context of two or more nucleic acid sequences, refer to two or more sequences or subsequences that are the same or have a specified percentage of amino acid residues or nucleotides that are the same, when compared and aligned for maximum correspondence, as measured using one of the following sequence comparison algorithms or by visual inspection.

The phrase “substantially identical,” in the context of two nucleic acids or polypeptides, refers to two or more sequences or subsequences that have at least about 60%, at least about 70%, preferably about 80%, most preferably about 90 to about 95% nucleotide residue identity, when compared and aligned for maximum correspondence, as measured using one of the following sequence comparison algorithms or by visual inspection.

For sequence comparison, typically one sequence acts as a reference sequence, to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally Ausubel, et al., supra).

Another example of algorithm that is suitable for determining percent sequence identity and sequence similarity is the BLAST algorithm, which is described in Altschul et al., J. Mol. Biol. 215:403-410 (1990). Software for performing BLAST analyses is publicly available through the National Center for Biotechnology Information (www.ncbi.nlm.nih.gov/). For identifying whether a nucleic acid is within the scope of the invention, the default parameters of the BLAST programs are suitable. The BLASTN program (for nucleotide sequences) uses as defaults a wordlength (W) of 11, an expectation (E) of 10, M=5, N=−4, and a comparison of both strands.

In addition to calculating percent sequence identity, the BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide sequences would occur by chance. For example, a nucleic acid is considered similar to a reference sequence if the smallest sum probability in a comparison of the test nucleic acid to the reference nucleic acid is less than about 0.1, more preferably less than about 0.01, and most preferably less than about 0.001.

A further indication that two nucleic acid sequences are substantially identical is that the two molecules hybridize to each other under stringent conditions and when the sequences are present in a complex mixture (e.g., total cellular) comprising non-complementary DNA or RNA. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology—Hybridization with Nucleic Probes, “Overview of principles of hybridization and the strategy of nucleic acid assays” (1993). Generally, stringent conditions are selected to be about 5-10° C. lower than the thermal melting point (Tₘ) for the specific sequence at a defined ionic strength pH. The Tₘ is the temperature (under defined ionic strength, pH, and nucleic concentration) at which 50% of a first sequence complementary to a second sequence hybridize to the second sequence at equilibrium (as the second sequences are present in excess, at Tₘ, 50% of the first sequence are occupied at equilibrium). Stringent conditions will be those in which the salt concentration is less than about 1.0 sodium ion, typically about 0.01 to 1.0 M sodium ion concentration (or other salts) at pH 7.0 to 8.3 and the temperature is at least about 30° C for short probes (e.g., 10 to 50 nucleotides) and at least about 60° C for long probes (e.g., greater than 50 nucleotides). Stringent conditions may also be achieved with the addition of destabilizing agents as formamide.

An example of highly stringent wash conditions is 0.15M NaCl at from 70 to 80° C. with 72° C. being preferable for about 15 minutes. An example of stringent wash conditions is a 0.2×SSC wash at about 60 to 70° C., preferably 65° C. for 15 minutes (see, Sambrook et al., Molecular Cloning, A Laboratory Manual, Cold Spring Harbor Publish., Cold Spring Harbor, N.Y. 2nd ed. (1989) for a description of SSC buffer). Often, a high stringency wash is preceded by a low stringency wash to remove background probe signal.

An exemplary medium stringency wash for a duplex of, e.g., more than 100 nucleotides, is 1×SSC at 40 to 50° C., preferably 45° C. for 15 minutes. An exemplary low stringency wash for a duplex of, e.g., more than 100 nucleotides, is 4-6×SSC at 35 to 45° C., with 40° C. being preferable, for 15 minutes. These conditions may be used to detect additional sequences that are “substantially identical” to the sequences disclosed herein.

Unless defined otherwise all technical and scientific terms used herein have the same meaning as commonly understood to one of ordinary skill in the art to which this invention belongs.

To determine the increased expression levels of gene sequences in the practice of the present invention, any method known in the art may be utilized. In one preferred embodiment of the invention, expression based on detection of RNA which hybridizes to a sequence disclosed herein is used. This is readily performed by any RNA detection or amplification+detection method known or recognized as equivalent in the art such as, but not limited to, reverse transcription-PCR, the methods disclosed in U.S. patent application Ser. No. 10/062,857 (filed on Oct. 25, 2001) as well as U.S. Provisional Patent Applications 60/298,847 (filed Jun. 15, 2001) and 60/257,801 (filed Dec. 22, 2000), and methods to detect the presence, or absence, of RNA stabilizing or destabilizing sequences.

A preferred embodiment using a nucleic acid based assay to determine expression is by immobilization of one or more sequences identified herein on a solid support, including, but not limited to, a solid substrate as an array or to beads or bead based technology as known in the art. Alternatively, solution based expression assays known in the art may also be used. The immobilized sequence(s) may be in the form of polynucleotides that are unique or otherwise specific to the sequence(s) such that the polynucleotide would be capable of hybridizing to a DNA or RNA corresponding to the sequence(s). These polynucleotides may be the full length of the sequence(s) or be short portions of the sequence(s) (up to one nucleotide shorter than the sequence by deletion from the 5′ or 3′ end of the sequence) that are optionally minimally interrupted (such as by mismatches or inserted non-complementary basepairs) such that hybridization with a DNA or RNA corresponding to the sequence(s) is not affected. Preferably, the polynucleotides used are from the 3′ end of the PR 3′ UTR as disclosed herein, such as within about 350, about 300, about 250, about 200, about 150, about 100, or about 50 nucleotides from the polyadenylation site as disclosed herein.

Alternatively, amplification of sequences from the 3′ end of the disclosed UTR sequences by methods such as quantitative PCR may be used to determine the expression levels of the sequences. The Ct values generated by such methods may be used to determine expression levels as described herein.

The immobilized sequence(s) may be used to determine the state of nucleic acid samples prepared from sample breast cell(s) for which the PR and/or ER status is not known or for confirmation of a status that is already assigned to the sample breast cell(s). Without limiting the invention, such a cell may be from a patient suspected of being afflicted with, or at risk of developing, breast cancer. Expression of PR transcript sequences as disclosed herein has been correlated with expression of PR protein in breast cells as determined by immunohistochemical (IHC) staining. The immobilized polynucleotide(s) need only be sufficient to specifically hybridize, optionally under stringent conditions, to the corresponding nucleic acid molecules derived from the sample.

The invention is preferably practiced with unique sequences present within the sequences disclosed herein. The uniqueness of a disclosed sequence refers to the portions or entireties of the sequences which are present to the exclusion in other, non-PR, genes. Preferred unique sequences for the practice of the invention are those which contribute to the consensus sequences for the 3′ UTR such that the unique sequences will be useful in detecting expression in a variety of individuals rather than being specific for a polymorphism present in some individuals. Alternatively, sequences unique to an individual or a subpopulation may be used. The preferred unique sequences are preferably of the lengths of primer and probe polynucleotides of the invention as discussed herein.

In particularly preferred embodiments of the invention, polynucleotides having sequences present in the 3′ end of the disclosed 3′ UTR are used to detect expression levels in breast cells. Alternative polynucleotides may contain sequences found in the 3′ portions of the PR coding region. Polynucleotides containing a combination of sequences from the coding and 3′ non-coding regions preferably have the sequences arranged contiguously, with no intervening heterologous, non-PR, sequence(s).

Preferred polynucleotides contain sequences from the disclosed 3′ UTR of at least about 16, at least about 18, at least about 20, at least about 22, at least about 24, at least about 26, at least about 28, at least about 30, at least about 32, at least about 34, at least about 36, at least about 38, at least about 40, at least about 42, at least about 44, or at least about 46 consecutive nucleotides. The term “about” as used in the previous sentence refers to an increase or decrease of 1 from the stated numerical value. Even more preferred are polynucleotides containing sequences of at least or about 50, at least or about 100, at least about or 150, at least or about 200, at least or about 250, at least or about 300, at least or about 350, or at least or about 400 consecutive nucleotides. The term “about” as used in the preceding sentence refers to an increase or decrease of 25 from the stated numerical value.

Polynucleotides combining the sequences from a 3′ untranslated and/or non-coding region and the associated 3′ end of the coding region are preferably at least or about 100, at least about or 750, at least or about 800, at least or about 850, at least or about 900, at least or about 950, or at least or about 1000 consecutive nucleotides. Polynucleotides containing mutations relative to the disclosed sequences may also be used so long as the presence of the mutations still allows hybridization to produce a detectable signal.

The above assay embodiments may be used in a number of different ways to identify or detect PR and/or ER expression status in a breast cancer cell sample from a patient. In many cases, this may reflect a secondary screen for the patient, who may have already undergone mammography or physical exam as a primary screen. If positive, the subsequent needle biopsy, ductal lavage, fine needle aspiration, or other analogous methods may provide the sample for use in the above assay embodiments. The present invention is particularly useful in combination with non-invasive protocols, such as ductal lavage or fine needle aspiration, to prepare a breast cell sample.

The present invention provides an objective set of criteria, in the form of gene expression profiles of a discrete set of genes, to discriminate (or delineate) between PR and/or ER positive and negative cells, such as cancer cells and those of breast cancer.

In one embodiment of the invention, the isolation and analysis of a breast cancer cell sample may be performed as follows:
(1) Ductal lavage or other non-invasive procedure is performed on a patient to obtain a sample.
(2) Sample is prepared and coated onto a microscope slide. Note that ductal lavage results in clusters of cells that are cytologically examined as stated above.
(3) Pathologist or image analysis software scans the sample for the presence of non-normal and/or atypical cells.
(4) If non-normal and/or atypical cells are observed, those cells are harvested (e.g. by microdissection such as LCM).
(5) RNA is extracted from the harvested cells.
(6) RNA is purified, amplified, and labeled.
(7) Labeled nucleic acid is contacted with a microarray containing sequence(s) disclosed herein under hybridization conditions to allow hybridization to occur, then processed and scanned to obtain an intensity (relative to a control for general gene expression in cells) which determines the level of expression of the sequence(s) in the cells.

Alternatively, quantitative PCR using primers (and optionally probes) as described herein is used to detect PR expression.

A specific example of the above method would be performing ductal lavage following a primary screen, observing and collecting non-normal and/or atypical cells for analysis.

With use of the present invention, skilled physicians may prescribe treatments based on non-invasive samples that they would have prescribed for a patient which had previously received a diagnosis via a solid tissue biopsy.

The above discussion is also applicable where a palpable lesion is detected followed by fine needle aspiration or needle biopsy of cells from the breast. The cells are plated and reviewed by a pathologist or automated imaging system which selects cells for analysis as described above.

The present invention may also be used, however, with solid tissue biopsies. For example, a solid biopsy may be collected and prepared for visualization followed by determination of expression of one or more sequences identified herein to determine PR and/or ER status in breast cancer. One preferred means is by use of in situ hybridization with polynucleotide or protein identifying probe(s) for assaying expression of said sequence(s).

In an alternative method, the solid tissue biopsy may be used to extract molecules followed by analysis for expression of one or more sequence(s). This provides the ability to leave out the need for visualization and collection of only those cells suspected of being non-normal and/or atypical. This method may of course be modified such that only cells suspected of being non-normal and/or atypical are collected and used to extract molecules for analysis. This would require visualization and selection as an prerequisite to expression analysis.

In a further modification of the above, both normal cells and cells suspected of being non-normal and/or atypical are collected and used to extract molecules for analysis of PR expression. The approach, benefits and results are as described above using non-invasive sampling.

Other uses of the present invention include providing the ability to identify samples containing cells, including cancer cells and breast cancer cells, as being those of PR and/or ER positive or negative for further research or study. This provides a particular advantage in many contexts requiring the identification of PR and/or ER status (such as in relation to breast cancer) based on objective genetic or molecular criteria.

The materials for use in the methods of the present invention are ideally suited for preparation of kits produced in accordance with well known procedures. The invention thus provides kits comprising agents for the detection of expression of the disclosed sequences for identifying PR and/or ER status as disclosed herein. Such kits optionally comprising the agents with an identifying description or label or instructions relating to their use in the methods of the present invention, is provided. Such a kit may comprise containers, each with one or more of the various reagents (typically in concentrated form) utilized in the methods, including, for example, pre-fabricated microarrays, buffers, the appropriate nucleotide triphosphates (e.g., dATP, dCTP, dGTP and dTTP; or rATP, rCTP, RGTP and UTP), reverse transcriptase, DNA polymerase, RNA polymerase, and one or more primer complexes of the present invention (e.g., appropriate length poly(T) or random primers linked to a promoter reactive with the RNA polymerase). A set of instructions will also typically be included.

The methods provided by the present invention may also be automated in whole or in part. All aspects of the present invention may also be practiced such that they consist essentially of the disclosed sequences to the exclusion of material irrelevant to the identification of PR and/or ER expression status.

Having now generally described the invention, the same will be more readily understood through reference to the following examples which are provided by way of illustration, and are not intended to be limiting of the present invention, unless specified.

### EXAMPLES

### Example I

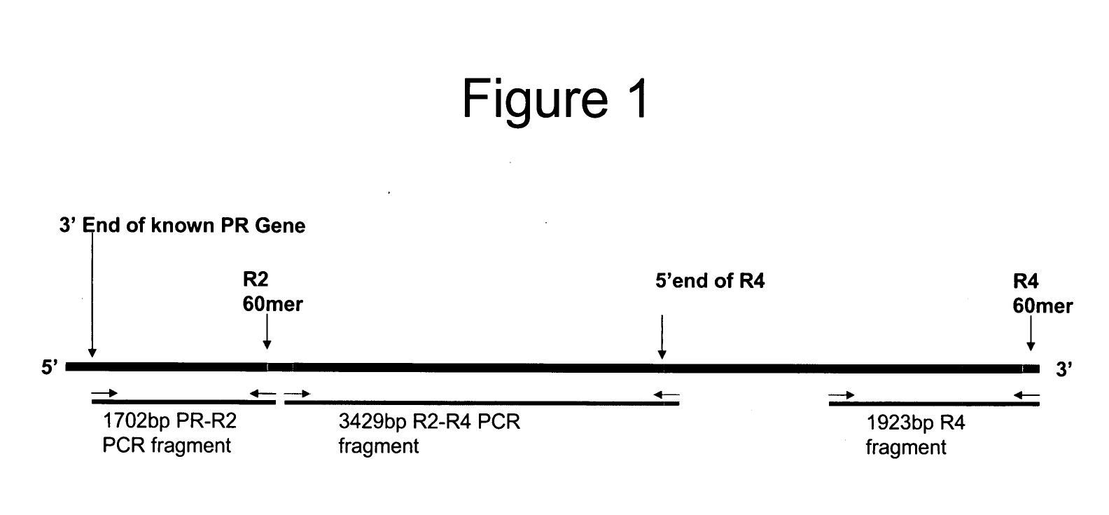

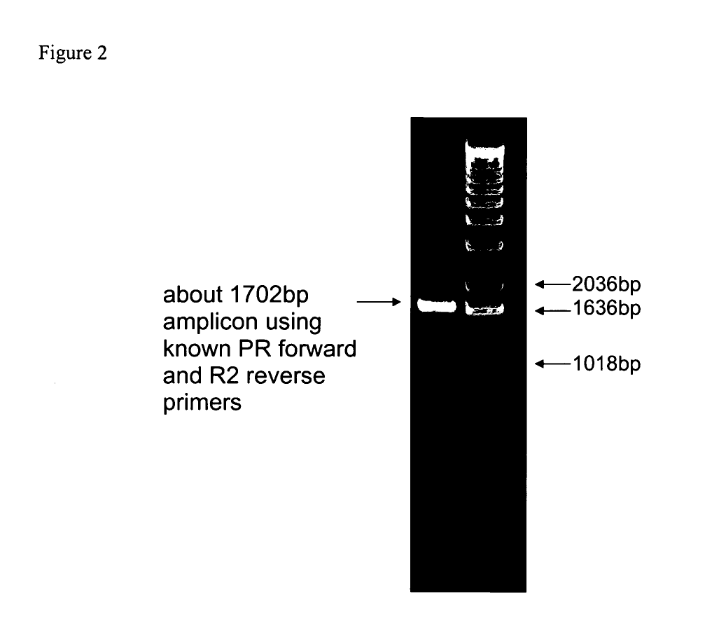

A link between the known PR sequences in X51730 and region 2 within positions 5004 to 13753 of SEQ ID NO:1 was confirmed by PCR amplification of a contiguous nucleic acid fragment of about 1702 bp as shown in FIG. 1. Briefly, a forward primer of sequence 5′-TTGTCCTCTAATGAGGTATTGCGAG-3′ (SEQ ID NO:5), which hybridizes within the known PR sequence, was used in combination with a reverse primer of sequence 5′-ATTGCAGACAGAGATCAACATCGA-3′ (SEQ ID NO:6), which hybridizes to a portion of region 2 as disclosed herein. As shown in FIG. 2, an amplified polynucleotide (amplicon) of the expected length of about 1702 bp (designated “1702” and suitable for isolation and biological deposit as well as further use as a probe or as an insert into a vector) was seen upon PCR amplification of expressed PR sequences using these primers. Accordingly, a physical link between the end of the known PR sequence and sequences within region 2 is established. The amplicon necessarily includes region 1 as disclosed herein.

In an analogous manner, an expected PCR amplicon of about 3429 bp corresponding to the sequence between region 2 and the 5′ end of region 4 of the disclosed UTR (as shown in FIG. 1) was obtained using a forward primer of sequence 5′-TATGGCTTCACCAAATGGAAA-3′ (SEQ ID NO:7) and a reverse primer of sequence 5′-TGTGAAAATCTCTTCCTATCCCTAAT-3′ (SEQ ID NO:8). The resulting amplified polynucleotide is designated “3429” and is suitable for isolation and biological deposit as well as further use as a probe or as an insert into a vector.

The two forward primers used above in this example were used in subsequent experiments as described in the examples below.

As shown in FIG. 1, an amplified polynucleotide designated “1923” suitable for isolation and biological deposit as well as further use as a probe or as an insert into a vector was produced by PCR using a forward primer of sequence 5′-ATCAGATGCCATTATCAAGTGGAATTA-3′ (SEQ ID NO:9), which hybridizes within region 4, was used in combination with a reverse primer of sequence 5′-CCAGCCAATTTAACATAAAACACTTG-3′ (SEQ ID NO:10), which hybridizes to a more 3′ portion of region 4. This reverse primer was used in subsequent experiments as described below.

### Example II

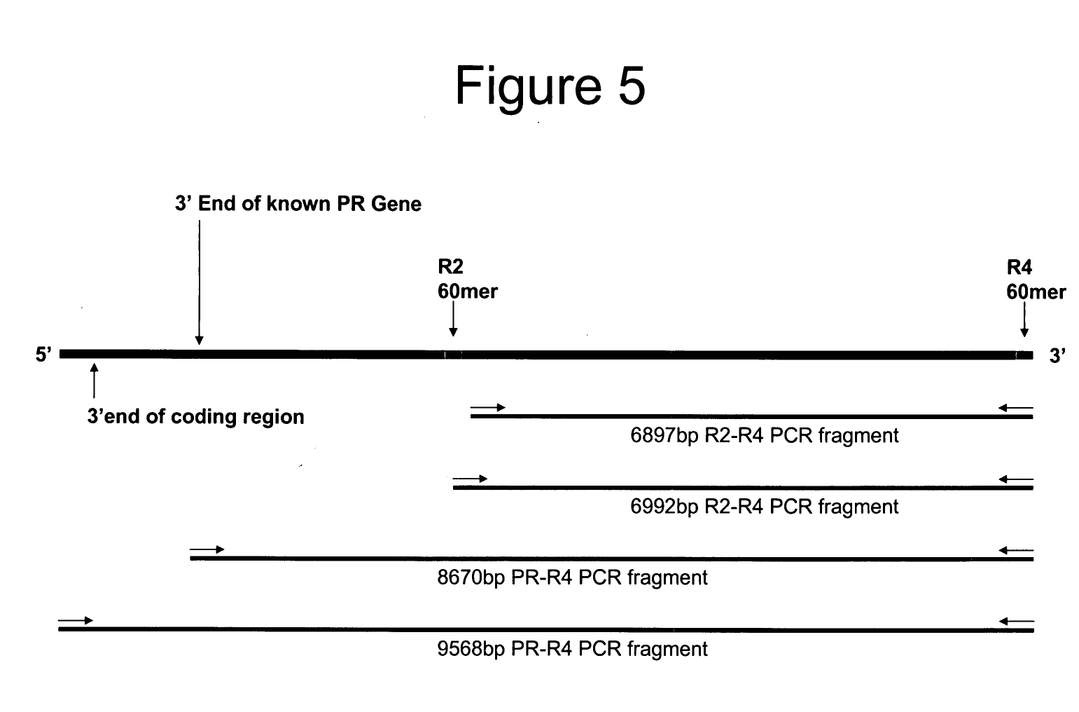

A link between the known PR sequences in X51730 and region 4 within positions 5004 to 13753 of SEQ ID NO:1 was confirmed by PCR amplification of two contiguous nucleic acid fragments as shown in FIG. 5. A first fragment from the PR coding region was obtained using a forward primer of sequence 5′-CAAAACTTCTTGATAACTTGCATGAT-3′ (SEQ ID NO:1 1), which hybridizes within the known PR coding region, and SEQ ID NO:10. The resulting amplified polynucleotide is designated “9568” and is suitable for isolation and biological deposit as well as further use as a probe or as an insert into a vector.

A second fragment from the 3′ end of the known PR gene was obtained using SEQ ID NO:5 and SEQ ID NO:10. The resulting amplified polynucleotide is designated “8670” and is suitable for isolation and biological deposit as well as further use as a probe or as an insert into a vector.

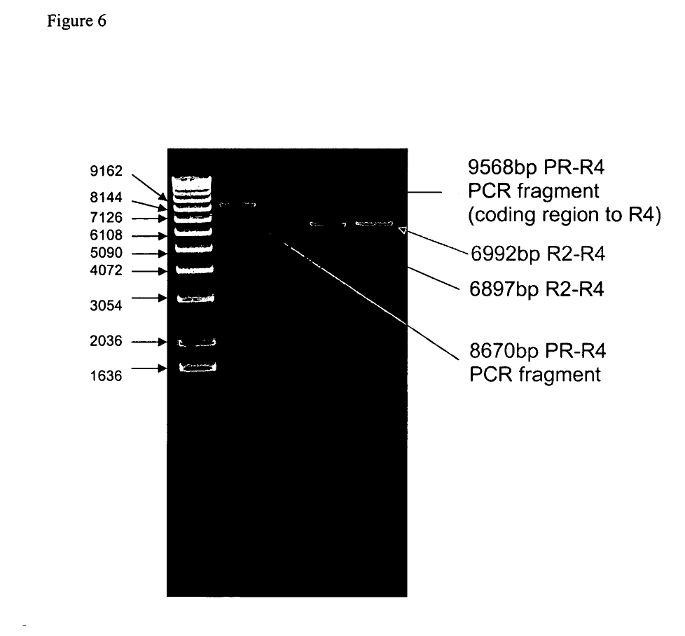

As shown in FIG. 6, amplicons of the expected lengths of about 9568 bp and about 8670 bp, respectively, were seen upon PCR amplification of expressed PR sequences using the above two pairs of primers. Accordingly, a physical link between the known PR sequence and sequences within region 4 is established. The amplicon necessarily includes regions 1, 2 and 3 as disclosed herein.

In an analogous manner, expected PCR amplicons of about 6897 bp and about 6992 bp corresponding to the sequence between regions 2 and 4 of the disclosed UTR (as shown in FIG. 1) were obtained using SEQ ID NOS:7 and 10 as one pair of forward and reverse primers, and 5′-TCGATGTTGATCTCTGTCTGCAAT-3′ (SEQ ID NO:12) and SEQ ID NO:10 as another pair, respectively. The resulting amplified polynucleotides are designated “6897” and “6992” and are suitable for isolation and biological deposit as well as further use as a probe or as an insert into a vector. FIG. 6 shows the relative molecular weight of these fragments.

### Example III

The invention may be practice by use of a quantitative PCR assay using a Sybr Green detection system. As an exemplification, a forward primer of sequence 5′-TGGTTCACATAAGAAAACAGTCTAC (SEQ ID NO:13), which hybridizes within 168 nucleotides of the 3′ end of SEQ ID NO:1, is used in combination with a reverse primer of sequence 5′-CATTTCAAACCACCAGCC (SEQ ID NO:14), which hybridizes within 114 nucleotides of the 3′ end of SEQ ID NO:1. The resulting amplified sequence is 72 bp long. Of course this exemplification can be practiced with primers that comprise the sequences of SEQ ID NOS:13 and 14 and additional nucleotides at the 5′ end of the primers.

Alternatively, the invention may be practiced by use of a quantitative PCR assay using a Taqman probe. As an exemplification, a forward primer of sequence 5′-AAGAAAACAGTCTACTCAGCTTGACA (SEQ ID NO:15), which hybridizes within 158 nucleotides of the 3′ end of SEQ ID NO:1, is used in combination with a reverse primer of sequence 5′-ATGTGAAGATGATTCATTTCAAACC (SEQ ID NO:16), which hybridizes within 107 nucleotides of the 3′ end of SEQ ID NO:1, and a labeled Taqman probe having the sequence 5′-TGTTTTATGTTAAATTGGCTGG (SEQ ID NO:17), which hybridizes to SEQ ID NO:1 between SEQ ID NOS:15 and 16. The resulting amplified sequence is 76 bp long. Of course this exemplification can be practiced with primers that comprise the sequences of SEQ ID NOS: 15 and 16 and additional nucleotides at the 5′ end of the primers.

All references cited herein, including patents, patent applications, and publications, are hereby incorporated by reference in their entireties, whether previously specifically incorporated or not.

Having now fully described this invention, it will be appreciated by those skilled in the art that the same can be performed within a wide range of equivalent parameters, concentrations, and conditions without departing from the spirit and scope of the invention and without undue experimentation.

While this invention has been described in connection with specific embodiments thereof, it will be understood that it is capable of further modifications. This application is intended to cover any variations, uses, or adaptations of the invention following, in general, the principles of the invention and including such departures from the present disclosure as come within known or customary practice within the art to which the invention pertains and as may be applied to the essential features hereinbefore set forth.

1. A polynucleotide comprising a progesterone receptor (PR) cDNA comprising all or part of positions 5004 to 13753, inclusive, of SEQ ID NO:1 and optionally
a) all or part of positions 1-5003, inclusive, of SEQ ID NO:1; or
b) all or part of the PR coding region.

2. A polynucleotide according to claim 1 comprising SEQ ID NO:1 or positions 5004 to 13753, inclusive, of SEQ ID NO:1.

3. A polynucleotide according to claim 1 comprising all or part of positions 5004 to 13753, inclusive, of SEQ ID NO:1 and at least 154 contiguous nucleotides of the PR coding region.

4. The polynucleotide according to claim 3, comprising at least 15 nucleotides of positions 5004 to 13753, inclusive, of SEQ ID NO:1.

5. A polynucleotide of about 15 to about 900 nucleotides in length and comprising about 15 to about 500 contiguous nucleotides of positions 5004 to 13753, inclusive, of SEQ ID NO:1.

6. An isolated polynucleotide comprising the human progesterone receptor 3′ untranslated region.

7. An isolated nucleic acid molecule which hybridizes to the polynucleotide of claim 3 under stringent conditions.

8. The nucleic acid molecule of claim 7 having a length of about 15 to about 8900 nucleotides.

9. An isolated nucleic acid molecule having a length from about 15 to about 8900 nucleotides which is at least 85% identical to the polynucleotide of claim 3.

10. An isolated nucleic acid molecule having a length from about 15 to about 8900 nucleotides which is at least 90% identical to the polynucleotide of claim 3.

11. An isolated nucleic acid molecule having a length from about 15 to about 8900 nucleotides which is at least 95% identical to the polynucleotide of claim 3.

12. A method of quantitative PCR analysis comprising quantitative PCR amplification of all or part of positions 5004 to 13753, inclusive, of SEQ ID NO:1.

13. The method of claim 12 wherein said amplification is of at least 50 nucleotides of positions 5004 to 13753, inclusive, of SEQ ID NO:1.

14. The method of claim 13 wherein said amplification is of at least 75 nucleotides of positions 5004 to 13753, inclusive, of SEQ ID NO:1.

15. The method of claim 14 wherein said amplification is of at least 100 nucleotides of positions 5004 to 13753, inclusive, of SEQ ID NO:1.

16. The method of claim 10 wherein said amplification is of at least 150 nucleotides of positions 5004 to 13753, inclusive, of SEQ ID NO:1.

17. The method of claim 12 wherein said analysis is of a biological sample for expression of all or part of positions 5004 to 13753, inclusive, of SEQ ID NO:1.

18. A pair of PCR primers for use in the method of claim 12.

19. The pair of PCR primers of claim 18, wherein said pair comprises two polynucleotides comprising the sequences of SEQ ID NOS: 13 and 14; and 15 and 16.

20. A method of preparing a polynucleotide containing all or part of the 3′ untranslated region of a progesterone receptor (PR) transcript, said method comprising PCR amplification using a first primer which hybridizes to all or part of positions 1 to 5003, inclusive, of SEQ ID NO:1 and a second primer which hybridizes to a sequence from within positions 5004 to 13753, inclusive, of SEQ ID NO:1 or which hybridizes to the region comprising the polyA tail of a PR transcript.

21. A polynucleotide prepared by the method of claim 20.

22. A method of preparing a polynucleotide containing all or part of the 3′ untranslated region of a progesterone receptor gene, said method comprising PCR amplification using a pair of primers which hybridize to all or part of positions 5004 to 13753, inclusive, of SEQ ID NO:1.

23. A method of detecting the expression of human PR, said method comprising obtaining a nucleic acid containing sample from a human subject; and
amplifying all or part of positions 5004 to 13753, inclusive, of SEQ ID NO:1 by quantitative PCR,
wherein detection of the amplified sequence is indicative of PR expression.

24. A method of detecting the expression of human PR, said method comprising
obtaining a nucleic acid containing sample from a human subject; and
detecting hybridization between a probe comprising at least 15 contiguous nucleotides of positions 5004 to 13753, inclusive, of SEQ ID NO:1 to the nucleic acids of said sample,
wherein either said sample or said probe is labeled with a detectable marker.
